(19) United States
(12) Patent Application Publication
(10) Pub. No.: US 2026/0258453 A1
(43) Pub. Date: Sep. 3, 2026

(54) METHOD FOR PRODUCING ALLITOL

(71) Applicant: ANNIKKI GMBH, Raaba-Grambach (AT)

(72) Inventors: Nicole STAUNIG, Raaba-Grambach (AT); Maria DUPONT, Raaba-Grambach (AT)

(21) Appl. No.: 19/474,599

(22) PCT Filed: Apr. 11, 2024
(86) PCT No.: PCT/EP2024/059807

§ 371 Date: Oct. 29, 2025

(30) Foreign Application Priority Data
Apr. 11, 2023 (EP) 23167347.6
Dec. 22, 2023 (EP) 23219719.4

(51) Int. Cl. C12P 7/18; C12N 9/02; C12N 9/04; C12N 9/90; C12P 19/02; C12P 19/24

(52) U.S. Cl. CPC C12P 7/18; C12N 9/0006; C12N 9/0093; C12N 9/90; C12P 19/02; C12P 19/24; C12Y 101/01001; C12Y 101/01002; C12Y 101/01047; C12Y 117/01; C12Y 501/03

(57) ABSTRACT

A method for producing allitol by forming in vitro D-psicose from D-fructose present in an aqueous solution by treatment with an epimerase, which D-psicose is reduced in vitro to allitol by treatment with an NAD(P)H-dependent oxidoreductase, while the NAD(P)⁺ resulting during reduction is enzymatically reduced to NAD(P)H again with a hydrogen donor, characterized in that a secondary alcohol is used as hydrogen donor. (FIG. 1)

The present invention relates to an enzymatic method producing the rare sugar alcohol allitol from D-fructose.

### BACKGROUND OF THE INVENTION

Allitol is a hexavalent sugar alcohol that rarely occurs in nature and has been detected, for example, in the leaves of sweetspire (Itea sp.) (Hough & Stacey, 1963). It is achiral and therefore forms an interface between D- and L-hexoses in the so-called Izumoring strategy (Izumori, 2006; Hassanin et al., 2017). Allitol can thus serve as a precursor for the production of D-psicose (Gullapalli et al., 2007; Poonperm et al., 2007) or L-psicose (Takeshita et al., 1996). In addition, allitol can also be used as a sweetener due to its sweet taste (Hassanin et al., 2017). WO 2020195106 A1 describes a possible use for allitol as an anti-adipose agent (slimming agent).

Allitol is obtainable from D-fructose in two chemical conversion steps: 1) epimerization of D-fructose to D-psicose (C3 epimer) and 2) reduction of D-psicose to allitol.

The first step is accomplished with a ketose-3-epimerase (Izumori et al., 1993). Ketose-3-epimerases can be divided into three groups according to substrate specificity: 1) D-tagatose-3-epimerase (DTE), 2) D-psicose-3-epimerase (DPE) or D-allulose-3-epimerase (DAE), and 3) L-ribulose-3-epimerase (LRE). During epimerization, an equilibrium ratio between the two epimers is established. Depending on the reaction conditions (temperature between 4° and 70° C., pH between 6 and 11), this ratio ranges between 80:20 and 62.5:37.5 (D-fructose: D-psicose). Many of the epimerases also require a divalent metal ion such as Mn²⁺ or Co²⁺ (toxic) as a co-factor (Zhang et al., 2016; Jiang et al., 2020).

The reduction of D-psicose to allitol can be carried out e.g. microbially, with Enterobacter agglomerans strain 221e (Muniruzzaman et al., 1995) or with Klebsiella oxytoca G4A4 (Han et al., 2014).

NAD-dependent ribitol dehydrogenase (RDH; EC 1.1.1.56), which is responsible for converting the pentavalent sugar alcohol ribitol to the ketopentose D-ribulose, also catalyzes the reduction of D-psicose to allitol.

Zhu et al. (2015) expressed D-psicose-3-epimerase from Ruminococcus sp. and RDH from Klebsiella oxytoca, a formate dehydrogenase (FDH) from Candida methylica (for regeneration of the co-factor nicotinamide adenine dinucleotide NADH; oxidizes formate to CO₂) and a glucose-fructose facilitator gene product from Zymomonas mobilis in Escherichia coli. With these modifications, the recombinant strain was able to produce 16.5 g/l of allitol from 18 g/l of D-fructose (92% conversion after 18 h).

Wen et al. (2022) also used a recombinant E. coli strain (with expressed RDH and FDH), that produced 58.5 g/l of allitol from 90 g/l of D-psicose in 1 hour.

Wang et al. (2023) describe an E. coli whole-cell biocatalyst for converting D-fructose to allitol. The E. coli cells used contained a DPE from Clostridiales, an RDH from Providencia alcalifaciens, an FDH from Starkeya, and another DPE from Rhizobium straminoryzae. In this way, D-fructose (500 mM=90 g/l) was converted to 452 mM of allitol within 12 hours at 37° C. and pH 6 using 1000 mM of sodium formate (two equivalents relative to D-fructose) and 0.5 mM of NAD⁺ (90.4% conversion). Approximately 30 mM of D-sorbitol (the reduction product of D-fructose) resulted as a by-product. The cells were separated by centrifugation and proteins that had leaked into the allitol-containing supernatant were deactivated by heat treatment.

An alternative enzymatic co-factor regeneration system, that is frequently used, is glucose dehydrogenase (GDH) with D-glucose as the substrate, which is oxidized to D-gluconolactone by NAD(P)⁺, thereby forming NAD(P)H. The lactone gets hydrolyzed in an aqueous environment to D-gluconic acid/D-gluconate.

Zhao et al. (2022) used a multi-enzyme self-assembly system consisting of DPE, RDH, and glucose dehydrogenase (GDH; for co-factor regeneration) in combination with a glucose isomerase in a whole-cell system to produce allitol (15 g/l) directly from D-glucose (25 g/l).

Feng et al. (2023) describe an in vivo process using an E. coli whole-cell catalyst with expressed DPE from Clostridium bolteae (requires the addition of 1 mM of Co²⁺), GDH from B. subtilis, and RDH from Providencia alcalifaciens for the simultaneous production of allitol and D-gluconic acid from D-fructose and D-glucose. Even with an E. coli whole-cell catalyst obtained under optimized fermentation conditions, only 10.6 g/l of D-gluconic acid from 25 g/l D-glucose and 9.8 g/l of allitol from 25 g/l of D-fructose could be produced within 12 hours, which the authors described as “uneconomical.” Further experiments with different concentrations of D-glucose (at a constant D-fructose concentration of 25 g/l) showed that the highest conversions (14.8 g/l of D-gluconic acid and 12 g/l of allitol) can be achieved with 20 g/l of D-glucose at the start of the reaction. This means, that at least ⅕ of the D-fructose used cannot be converted to allitol. Feng et al. subsequently used sugar cane molasses, which had been pretreated for the hydrolysis of sucrose using sulfuric acid and ultrasound, as a substrate. Within 12 hours, 42.7 g/l of allitol (30.7% conversion) and 56.2 g/l of D-gluconic acid (37.7% conversion) were obtained from 139.2 g/l of D-fructose and 149.1 g/l of D-glucose, which, in the opinion of the authors, could be due to the insufficient amount of biomass. In addition to crystallization, (sometimes costly and laborious) methods such as membrane separation processes like nanofiltration or simulated moving bed chromatography (SMBC) are proposed for separating the mixture of products and substrates.

For the production of allitol also cell-free processes have been described.

Hassanin et al. (2016) used a ribitol dehydrogenase (RDH) from Providencia alcalifaciens RIMD 1656011 and an FDH from Ogataea parapolymorpha DL-1 in the form of cell lysates to convert D-psicose (10 g/l) to allitol (94% in 6 h; addition of 2 mM of NAD⁺). An HPLC method was used to obtain allitol—the collected eluate was freeze-dried.

The regeneration of co-factors using alcohol dehydrogenases is known, e.g., from EP 2812439 B1 or described in Xu et al. (2021).

Takeshita et al. (2000) use a formate dehydrogenase (FDH) to regenerate the co-factor. A DTE from Pseudomonas cichorii ST-24 and an RDH from Klebsiella pneumoniae strain X22 (a mutant of K. pneumoniae IFO 3321) in the form of cell lysates are used to convert D-fructose (10 g/l) to allitol with the addition of 2.5 mM of NAD⁺ (100% conversion) in 48 hours. Pure allitol was obtained from the product solution by treating the reaction mixture with activated carbon, centrifugation/filtration, followed by deionization using ion exchange resins. Takeshita et al. (2000) further describe an optimization of the process for producing allitol from D-fructose. Despite this optimization, however, a disadvantage of this process is, that D-fructose can only be converted in relatively low concentrations of about 10 g/l.

This is where the object of the present invention comes in, with the aim of providing a method for producing allitol that improves the above-mentioned methods of Takeshita et al. (2000) and Feng et al. (2023) and, in particular, allows D-fructose to be used in higher substrate concentrations and achieves higher conversion rates.

### DETAILED DESCRIPTION OF THE INVENTION

According to the invention, the object is achieved by forming D-psicose through treatment with an epimerase in vitro from D-fructose, which is present in an aqueous solution, and where D-psicose is reduced in vitro to allitol by treatment with an NAD(P)H-dependent oxidoreductase, whereby the NAD(P)⁺, resulting during reduction, is enzymatically reduced again to NAD(P)H with a hydrogen donor, characterized in that a secondary alcohol is used as hydrogen donor.

Key features of the present invention are that the method is carried out in vitro, i.e., not fermentatively, and that a secondary alcohol is used as hydrogen donor.

In the sense of the present description and claims, a secondary alcohol is an organic compound with a secondary alcohol moiety.

It has been shown that with the inventive use of a secondary alcohol as hydrogen donor—instead of formic acid (formate) as hydrogen donor—D-fructose can be used in a much higher substrate concentration than according to the state of the art.

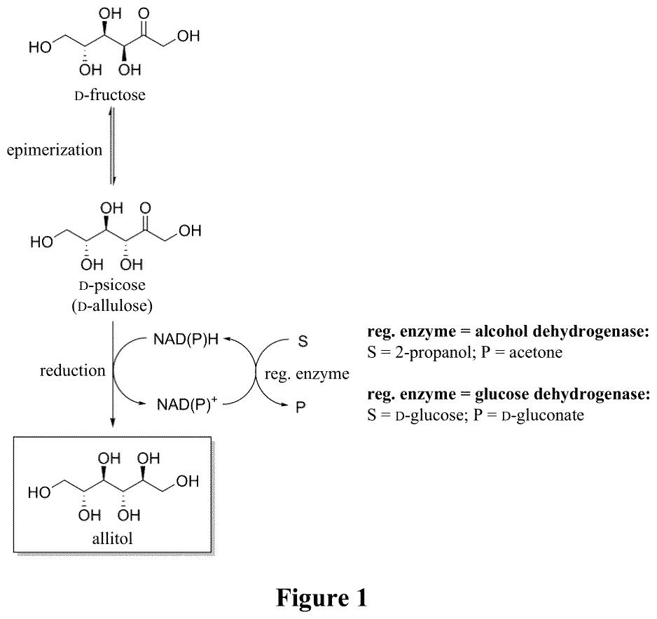

In a first step, D-psicose is formed in vitro from D-fructose by treatment with an epimerase and where D-psicose is reduced in vitro to allitol with an NAD(P)H-dependent oxidoreductase. Subsequently, allitol can be separated from the solution by crystallization. The inventive method is schematically shown in the accompanying FIG. 1.

A preferred variant of the inventive method consists in the secondary alcohol being 2-propanol (isopropanol). 2-Propanol is a very inexpensive hydrogen donor for the regeneration of NAD(P)H, and the oxidation product acetone is easily separable due to its volatility (Xu et al., 2021). The acetone recovered from the exhaust gas stream can be heterogeneously-catalytically rehydrogenated to 2-propanol (Al-Rabiah et al., 2022), either in the gas phase, in solution, or in isopropanol/acetone/water mixtures, and in future, there could be an increased focus on hydrogen from sustainable sources (“green hydrogen”). In addition, the reduction of acetone to 2-propanol can also be achieved enzymatically by means of alcohol dehydrogenase (in combination with an enzymatic oxidation).

A further preferred variant of the inventive method consists in the oxidized co-factor NAD(P)⁺ resulting from the reduction of D-psicose to allitol being reduced by means of a glucose dehydrogenase (GDH) and D-glucose with the formation of D-gluconolactone, which hydrolyzes, depending on the pH value in the aqueous environment, to D-gluconic acid or D-gluconate. D-gluconic acid is an important industrial chemical because of its wide range of possible applications, including as metal pickling agent (D-gluconic acid), acidifier (D-gluconic acid and D-gluconolactone), rust remover (sodium gluconate), or dietary supplement (calcium gluconate, magnesium gluconate, ferrous gluconate) (Kornecki et al., 2020; EP 0132557 B1).

The use of the co-factor regeneration systems described above (D-glucose+GDH as well as 2-propanol+ADH) avoids the emission of climate-damaging CO₂ and also large amounts of waste (such as non-reacted sodium formate in the FDH regeneration system) because, on the one hand, the products can be used in further applications (D-gluconate), and on the other hand, they can be chemically-catalytically (hydrogenation) or enzymatically regenerated (acetone to 2-propanol).

A further preferred variant of the inventive method is characterized, in that it is carried out as a one-pot reaction without isolation of the D-psicose.

The particularly preferred concentration of D-fructose is 100-250 g/l.

The particularly preferred concentration of D-glucose is 100-250 g/l.

The particularly preferred temperature range for the inventive method is between 25 and 45° C.

The inventive method is preferably carried out in a pH range between 6.0 and 9.0, most preferably between 7.0 and 8.5.

In a further preferred variant of the inventive method, the enzymes are present in a suspension and/or in the homogenate and/or in the lysate of the respective cells forming them, whereby lysates being particularly preferred.

In the sense of the present description and claims, suspension means a suspension of resting cells. These are harvested after cultivation (separated from the culture medium) and suspended in a suitable buffer system. Contrary to fermentative methods where whole cells can be used as well, resting cells cannot grow anymore due to the removal of carbon sources and nutrients, but only serve for reacting substrates (Lin & Tao, 2017). In this context, homogenate refers to a physically and/or chemically treated suspension (e.g., by means of pressure, lysozymes, or ultrasound), with the cell components being released from the cells. A lysate is obtained, when the insoluble cell components of the homogenate are, for example, removed by filtration or centrifugation (see Production of the enzymes & preparation of the lysates for details).

In a further variant, the enzymes can also be modified at the N-terminus with a water-soluble polymer such as polyethylene glycol, be immobilized in or on a solid matrix, or be part of a fusion protein.

In a further variant, the enzymes can be present in a powder, lyophilized, or spray-dried form.

In a particularly preferred embodiment of the method, only enzymes from the enzyme groups of epimerases and oxidoreductases are used for converting the starting material, wherein one or more of these enzymes are selected from each of these groups.

The epimerase used in the method can be from one of the EC 5.1.3.30 (D-psicose-3-epimerase) or EC 5.1.3.31 (D-tagatose-3-epimerase/L-ribulose-3-epimerase) groups, the first one mentioned being particularly preferred.

The enzyme used for the reduction of D-psicose belongs to the group of oxidoreductases, a short-chain dehydrogenase/reductase being particularly preferred.

The NAD(P)H-dependent oxidoreductase for the reduction of D-psicose to allitol preferably comprises or consists of an amino acid sequence selected from the group consisting of:
i) an amino acid sequence having at least 80% identity to SEQ ID NO: 2, SEQ ID NO: 8, or SEQ ID NO: 10,
ii) an amino acid sequence encoded by a nucleic acid having at least 80% identity to SEQ ID NO: 1, SEQ ID NO: 7, or SEQ ID NO: 9, and
iii) an amino acid sequence encoded by a nucleic acid that, under stringent conditions, binds to a nucleic acid molecule with the nucleic acid sequence of SEQ ID NO: 1, SEQ ID NO: 7, or SEQ ID NO: 9.

In general, an oxidoreductase with an amino acid sequence having at least 80% identity to SEQ ID NO: 2, SEQ ID NO: 8, or SEQ ID NO: 10 or being encoded by a nucleic acid having at least 80% identity to SEQ ID NO: 1, SEQ ID NO: 7, or SEQ ID NO: 9 or binding, under stringent conditions, to a nucleic acid molecule with the nucleic acid sequence of SEQ ID NO: 1, SEQ ID NO: 7, or SEQ ID NO: 9 is particularly suitable for the reduction of D-psicose to allitol.

SEQ ID NO: 1:
ATGACACAGTCTCTTCAGGGCAAGATCGTCGCCATTACTGGCGCGGCTTCGGGCATTGGCCTCGAATGC
GCCCGCTATCTCATCGAAGCTGGCGCGGTGGTCTATCTTCTGGACCGTGACGCCAAAACTCTCGAAGAC
AAAACGGCAGAACTCGGCAGCCAGGCCCATGCGATCATCGTCGATCTCTTCGACTACAAAACCGTAGAT
GCTGCGGTCGCGCAGATCGTTGAGGAGCAGGGCCGGATCGATGTTTTCCACGCCAACGCCGGCGCGTA
TGTGGGCGGCAATGTCTGGGAAGGGGATCCCGATAGCTGGGATGCGATGCTGCACCTGAACATCAAT
GCAGCTTTCCGCTCGGTCCGTGCCGTGCTGCCGCAGATGATGAAGCAGGAAAGTGGCGATATCGTCAT
GACCAGCTCGATCGCGGGCATGATCCCCATCATGGCCGAGCCGATCTACACGGCCTCGAAACATGCCG
TGCAGGCATTCGTACATACGGTGCGCCGACAGGTCGCGAAGTATGGAATTCGTGTGGGTGCAATCCAG
CCTGGTCCTGTAGTCACGCCTTTGCTGAAAGACTGGGATCAGGCCCGTCTTGAAGCCAACATCAAAGCG
GGTGCCCTGATGGAAGCCAAGGAAGTCGCCGAAGCCCTGATCTTCATCCTGACACGTTCAAAGGGTGT
CATGGTGCGGGATCTGGTCGTTCTGCCACATAACTTCGACGCCTAAGCTTAAGCGGCCGCACTCGAGCA
CCACCACCACCACCACTGAGATCCGGCTGCTAA
SEQ ID NO: 2:
MTQSLQGKIVAITGAASGIGLECARYLIEAGAVVYLLDRDAKTLEDKTAELGSQAHAIIVDLFDYKTVDAAVA
QIVEEQGRIDVFHANAGAYVGGNVWEGDPDSWDAMLHLNINAAFRSVRAVLPQMMKQESGDIVMTSSI
AGMIPIMAEPIYTASKHAVQAFVHTVRRQVAKYGIRVGAIQPGPVVTPLLKDWDQARLEANIKAGALMEA
KEVAEALIFILTRSKGVMVRDLVVLPHNFDA
SEQ ID NO: 7:
ATGACCTCTCCTCTCCAGGGTAAGATAGCCGCCATCACGGGCGGGGCTTCGGGCATCGGCCTCGAATG
TGTCCGCCAGATCGCCGCAAGTGGTGCCACGGTTTATATTCTCGACCGCGACCATCAGGCGCTCGACAA
GGCGCGCGAAGAATTGGGCGAGCGCGTTCATACCATCGAGGTCGATCTCTTCCGTTACGAAACGGTCG
ATCGCGCCATCGAAACCATCGTGTCCGAACAAGGACGCATCGACATTCTCCATGTCAATGCGGGCGCGT
ATATCGGCGGCAATGTCTGGGAAGGCGATCCCGATAAATGGGACAAGATGCTGAATCTCAACATCAAC
GCCGCCTTCCGTTCCGCCCGCGCCGTCATGCCCGCCATGATGAAGCAGAAAAGCGGCGATATCATCATG
ACAAGCTCGATCGCAGGCATCGTCCCGATCCCGGCGGAGCCGATCTACACGGCTTCCAAACATGCGGT
GCAAGCCTTCGCTCACACCATACGCCGCCAGTTGGCCCCGTTCGGCATCCGCGTCGGCGCCATCCAGCC
CGGCCCGGTCGTCACGCCCTTGCTCAATGATTGGGACCCCGAGCGCCTTAAAGCCAATATCGAGGCTG
GCGCCATGATGCAGCCTTCTGACGTCGCCGAAGCCGTGGTTTTCATGCTGTCCCGCCGCAAGGGAACG
GTAATCCGCGACTTGGTTCTGTTACCCCATTCTTTCGACGTCTAA
SEQ ID NO: 8:
MTSPLQGKIAAITGGASGIGLECVRQIAASGATVYILDRDHQALDKAREELGERVHTIEVDLFRYETVDRAIET
IVSEQGRIDILHVNAGAYIGGNVWEGDPDKWDKMLNLNINAAFRSARAVMPAMMKQKSGDIIMTSSIAGI
VPIPAEPIYTASKHAVQAFAHTIRRQLAPFGIRVGAIQPGPVVTPLLNDWDPERLKANIEAGAMMQPSDVA
EAVVFMLSRRKGTVIRDLVLLPHSFDV
SEQ ID NO: 9:
ATGGCTATATCTCTCGAAAACAACGTAGCTGCAATTACAGGTGCCGCTTCAGGTATCGGTCTCGAATGT
GCACGCACACTGATCAAAGCAGGCGCTAAAGTTGTCCTCATTGACCGAGCAGAAGATAGACTAAATCA
ATTGGTCGCAGAATTAGGTGAAAATGCAATTCCATTAGTTATCGATTTAATGAAACCAGAACAAGTCGA
TGGCATGTTAGCGCGTATTATCGAAAAGGCAGGCAGATTAGATATCTTTCATGCTAATGCTGGAGCTTA
CATTGGTGGGCCCGTAGCCGAAGGCGATCCCGATGTTTGGGATAAAGTCCTAAATTTAAATGTTAATGC
CGCATTCCGCTGTGTTCGCGCAGTTCTACCACACTTTATCGCACAAAAGTCAGGCGATATTCTATTCACC
AGCTCTATCGCTGGTATGGTTCCCGTAATTTGGGAGCCTATTTACACGGCATCAAAATTTGCGGTTCAA
GCATTCGTTCATTCTACTCGCCGTCAGGTTTCTGAACACGGTGTCCGTGTTGGTGCTGTATTACCTGGTC
CTGTTGTTACTGCGTTATTAGATGATTGGCCAAAAGAAAAACTTGAAGAAGCTTTAGCTAACGGTAGTT
TAATGCAACCCATTGAAGTTGCTGAGGCTGTTCTATTCATGCTGACGCGTCCAAGAAATATTACAATTCG
CGATTTAGTTATTTTACCCAATAGTGTTGACCTCTAA
SEQ ID NO: 10:
MAISLENNVAAITGAASGIGLECARTLIKAGAKVVLIDRAEDRLNQLVAELGENAIPLVIDLMKPEQVDGMLA
RIIEKAGRLDIFHANAGAYIGGPVAEGDPDVWDKVLNLNVNAAFRCVRAVLPHFIAQKSGDILFTSSIAGMV
PVIWEPIYTASKFAVQAFVHSTRRQVSEHGVRVGAVLPGPVVTALLDDWPKEKLEEALANGSLMQPIEVAE
AVLFMLTRPRNITIRDLVILPNSVDL

The oxidoreductases mentioned herein for the reduction of D-psicose to allitol preferably comprise or consist of an amino acid sequence having at least 80% identity to SEQ ID NO: 2, SEQ ID NO: 8, or SEQ ID NO: 10, more preferably 85%, even more preferably 90%, even more preferably 95%, even more preferably 98%, even more preferably 99%, most preferably 100%. Particularly preferably, the inventive oxidoreductase for the reduction of D-psicose to allitol comprises or consists of the amino acid sequence of SEQ ID NO: 2, SEQ ID NO: 8, or SEQ ID NO: 10.

Alternatively, the oxidoreductases for the reduction of D-psicose to allitol preferably comprise or consist of an amino acid sequence encoded by a nucleic acid having at least 80% identity to SEQ ID NO: 1, SEQ ID NO: 7, or SEQ ID NO: 9, more preferably 85%, even more preferably 90%, even more preferably 95%, even more preferably 98%, even more preferably 99%, most preferably 100%. Particularly preferably, the nucleic acid encoding the inventive oxidoreductase for the reduction of D-psicose to allitol comprises or consists of the nucleic acid sequence of SEQ ID NO: 1, SEQ ID NO: 7, or SEQ ID NO: 9.

The term “identity”, as used herein, refers to the percentage of identical nucleotides or amino acids between at least two nucleotide or amino acid sequences aligned using a standardized algorithm (“alignment”). Such an algorithm can, in a standardized and reproducible manner, insert gaps into the compared sequences to optimize the alignment between to sequences and thus achieve a more meaningful comparison of the two sequences.

The percentage identity between sequences can be determined by using one or more computer algorithms or programs known in the state of the art or described herein. According to the invention, the Basic Local Alignment Search Tool (BLAST) (Altschul et al., 1990) provided by the National Center for Biotechnology Information (NCBI) is used to determine identity. The BLAST software suite comprises several programs, including a tool called “BLAST 2 Sequences” which is used for the direct pairwise comparison of two nucleotide or amino acid sequences. “BLAST 2 Sequences” can also be retrieved interactively on the Internet via the NCBI World Wide Web page and used. The blastn program (for nucleotide sequences) uses as defaults a Word size (W) of 11, an Expected value (E) of 10, M=5, N=−4, and a comparison of both strands. For amino acid sequences, the blastp program uses as defaults a Word size of 3 and an Expected value (E) of 10 as default, and the BLOSUM62 scoring matrix (Henikoff & Henikoff, 1989), alignments (B) of 50, Expect value (E) of 10, M=5, N=−4.

Alternatively, the oxidoreductases for the reduction of D-psicose to allitol preferably comprise an amino acid sequence encoded by a nucleic acid that, under stringent conditions, binds to a nucleic acid molecule with the nucleic acid sequence of SEQ ID NO: 1, SEQ ID NO: 7, or SEQ ID NO: 9. As used herein, stringent conditions refer to conditions under which so-called specific hybrids, but no non-specific hybrids are formed. For example, stringent conditions comprise hybridization in 6×SSC (sodium chloride/sodium citrate) at 45° C. and then washing with 0.2 to 1×SSC, 0.1% SDS at 50 to 65° C.; or such conditions may include hybridization in 1×SSC at 65 to 70° C. and then washing with 0.3×SSC at 65 to 70° C. Hybridization can be carried out by conventionally known methods, such as those described by J. Sambrook et al. in Molecular Cloning, A Laboratory Manual, 2nd Ed., Cold Spring Harbor Laboratory (1989).

One aspect of the present invention relates to the use of an oxidoreductase for the reduction of D-psicose to allitol, wherein the oxidoreductase comprises or consists of an amino acid sequence selected from the group consisting of:
i) an amino acid sequence having at least 80% identity to SEQ ID NO: 2, SEQ ID NO: 8, or SEQ ID NO: 10,
ii) an amino acid sequence encoded by a nucleic acid having at least 80% identity to SEQ ID NO: 1, SEQ ID NO: 7, or SEQ ID NO: 9, and
iii) an amino acid sequence encoded by a nucleic acid that, under stringent conditions, binds to a nucleic acid molecule with the nucleic acid sequence of SEQ ID NO: 1, SEQ ID NO: 7, or SEQ ID NO: 9.

The alcohol dehydrogenase (ADH) used for co-factor regeneration can be from one of the groups EC 1.1.1.1 (NAD-dependent ADH) and EC 1.1.1.2 (NADP-independent ADH).

The NAD(P-dependent alcohol dehydrogenase for co-factor regeneration preferably comprises or consists of an amino acid sequence selected from the group consisting of:
i) an amino acid sequence having at least 80% identity to SEQ ID NO: 4,
ii) an amino acid sequence encoded by a nucleic acid having at least 80% identity to SEQ ID NO: 3, and
iii) an amino acid sequence encoded by a nucleic acid that, under stringent conditions, binds to a nucleic acid molecule with the nucleic acid sequence of SEQ ID NO: 3.

In general, an alcohol dehydrogenase with an amino acid sequence having at least 80% identity to SEQ ID NO: 4 or being encoded by a nucleic acid having at least 80% identity to SEQ ID NO: 3 or binding, under stringent conditions, to a nucleic acid molecule with the nucleic acid sequence of SEQ ID NO: 3 is particularly suitable for co-factor regeneration.

SEQ ID NO: 3:
ATGAAAGCTGCAGTTGTGGAACAATTTAAAAAGCCGTTACAAGTGAAAGAAGTGGAAAAACCTAAGAT
CTCATACGGGGAAGTATTAGTGCGCATCAAAGCGTGTGGGGTATGCCATACAGACTTGCATGCCGCAC
ATGGCGACTGGCCTGTAAAGCCTAAACTGCCTCTCATTCCTGGCCATGAAGGCGTCGGTGTAATTGAAG
AAGTAGGTCCTGGGGTAACACATTTAAAAGTTGGAGATCGCGTAGGTATCCCTTGGCTTTATTCGGCGT
GCGGTCATTGTGACTATTGCTTAAGCGGACAAGAAACATTATGCGAACGTCAACAAAACGCTGGCTATT
CCGTCGATGGTGGTTATGCTGAATATTGCCGTGCTGCAGCCGATTATGTCGTAAAAATTCCTGATAACTT
ATCGTTTGAAGAAGCCGCTCCAATCTTTTGCGCTGGTGTAACAACATATAAAGCGCTCAAAGTAACAGG
CGCAAAACCAGGTGAATGGGTAGCCATTTACGGTATCGGCGGGCTTGGACATGTCGCAGTCCAATACG
CAAAGGCGATGGGGTTAAACGTCGTTGCTGTCGATTTAGGTGATGAAAAACTTGAGCTTGCTAAACAA
CTTGGTGCAGATCTTGTCGTCAATCCGAAACATGATGATGCAGCACAATGGATAAAAGAAAAAGTGGG
CGGTGTGCATGCGACTGTCGTCACAGCTGTTTCAAAAGCCGCGTTCGAATCAGCCTACAAATCCATTCG
TCGCGGTGGTGCTTGCGTACTCGTCGGATTACCGCCGGAAGAAATACCTATTCCAATTTTCGATACAGT
ATTAAATGGAGTAAAAATTATTGGTTCTATCGTTGGTACGCGCAAAGACTTACAAGAGGCACTTCAATT
TGCAGCAGAAGGAAAAGTAAAAACAATTGTCGAAGTGCAACCGCTTGAAAACATTAACGACGTATTCG
ATCGTATGTTAAAAGGGCAAATTAACGGCCGCGTCGTGTTAAAAGTAGATTAA
SEQ ID NO: 4:
MKAAVVEQFKKPLQVKEVEKPKISYGEVLVRIKACGVCHTDLHAAHGDWPVKPKLPLIPGHEGVGVIEEV
GPGVTHLKVGDRVGIPWLYSACGHCDYCLSGQETLCERQQNAGYSVDGGYAEYCRAAADYVVKIPDNLS
FEEAAPIFCAGVTTYKALKVTGAKPGEWVAIYGIGGLGHVAVQYAKAMGLNVVAVDLGDEKLELAKQLG
ADLVVNPKHDDAAQWIKEKVGGVHATVVTAVSKAAFESAYKSIRRGGACVLVGLPPEEIPIPIFDTVLNGV
KIIGSIVGTRKDLQEALQFAAEGKVKTIVEVQPLENINDVFDRMLKGQINGRVVLKVD

The alcohol dehydrogenase mentioned herein for co-factor regeneration preferably comprises an amino acid sequence having at least 80% identity to SEQ ID NO: 4, more preferably 85%, even more preferably 90%, even more preferably 95%, even more preferably 98%, even more preferably 99%, most preferably 100%. Particularly preferably, the inventive alcohol dehydrogenase for co-factor regeneration comprises or consists of the amino acid sequence of SEQ ID NO: 4.

Alternatively, the alcohol dehydrogenase for co-factor regeneration preferably comprises or consists of an amino acid sequence encoded by a nucleic acid having at least 80% identity to SEQ ID NO: 3, more preferably 85%, even more preferably 90%, even more preferably 95%, even more preferably 98%, even more preferably 99%, most preferably 100%. Particularly preferably, the nucleic acid encoded by the inventive alcohol dehydrogenase for co-factor regeneration comprises or consists of the nucleic acid sequence of SEQ ID NO: 3.

One aspect of the present invention relates to the use of an alcohol dehydrogenase for co-factor regeneration, wherein the alcohol dehydrogenase comprises or consists of an amino acid sequence selected from the group consisting of:
i) an amino acid sequence having at least 80% identity to SEQ ID NO: 4,
ii) an amino acid sequence encoded by a nucleic acid having at least 80% identity to SEQ ID NO: 3 and
iii) an amino acid sequence encoded by a nucleic acid that, under stringent conditions, binds to a nucleic acid molecule with the nucleic acid sequence of SEQ ID NO: 3.

The glucose dehydrogenase (GDH) used for co-factor regeneration can be from one of the groups EC 1.1.1.47 (glucose-1-dehydrogenase), EC 1.1.1.118 (glucose-1-dehydrogenase (NAD⁺)), EC 1.1.1.119 (glucose-1-dehydrogenase (NADP⁺)), or EC 1.1.1.360 (glucose/galactose-1-dehydrogenase).

The NAD(P)-dependent glucose dehydrogenase for co-factor regeneration preferably comprises or consists of an amino acid sequence selected from the group consisting of:
i) an amino acid sequence having at least 80% identity to SEQ ID NO: 6,
ii) an amino acid sequence encoded by a nucleic acid having at least 80% identity to SEQ ID NO: 5, and
iii) an amino acid sequence encoded by a nucleic acid that, under stringent conditions, binds to a nucleic acid molecule with the nucleic acid sequence of SEQ ID NO: 5.

In general, a glucose dehydrogenase with an amino acid sequence having at least 80% identity to SEQ ID NO: 6 or being encoded by a nucleic acid having at least 80% identity to SEQ ID NO: 5 or binding, under stringent conditions, to a nucleic acid molecule with the nucleic acid sequence of SEQ ID NO: 5 is particularly suitable for co-factor regeneration.

SEQ ID NO: 5:
ATGTATACAGATTTAAAAGATAAAGTAGTTGTAATTACAGGTGGATCAACAGGTTTAGGACGCGCAA
TGGCTGTTCGTTTCGGTCAAGAAGAAGCAAAAGTTGTTATTAACTATTACAACAATGAAGAAGAAGCT
TTAGATGCGAAAAAAGAAGTAGAAGAAGCAGGCGGACAAGCAATCATCGTTCAAGGCGACGTAACA
AAAGAAGAAGATGTTGTAAACCTTGTTCAAACAGCTATTAAAGAATTCGGTACATTAGACGTTATGAT
TAATAACGCTGGTGTTGAAAACCCAGTTCCTTCTCATGAGTTATCTTTAGACAACTGGAATAAAGTTAT
TGATACAAACTTAACAGGTGCATTCTTAGGAAGCCGTGAAGCAATCAAATATTTTGTTGAAAACGACA
TTAAAGGAAACGTTATTAACATGTCTAGTGTTCATGAAATGATTCCTTGGCCATTATTTGTTCATTACG
CAGCAAGTAAAGGCGGTATGAAACTAATGACGGAAACATTGGCTCTTGAATATGCGCCAAAAGGTAT
CCGCGTAAATAACATTGGACCAGGTGCGATGAACACACCAATTAACGCAGAGAAATTTGCAGATCCT
GTACAACGTGCAGACGTAGAAAGCATGATTCCAATGGGTTACATCGGTAAACCAGAAGAAGTAGCA
GCAGTTGCAGCATTCTTAGCATCATCACAAGCAAGCTATGTAACAGGTATTACATTATTTGCTGATGG
TGGTATGACGAAATACCCTTCTTTCCAAGCAGGAAGAGGCTAA
SEQ ID NO: 6:
MYTDLKDKVVVITGGSTGLGRAMAVRFGQEEAKVVINYYNNEEEALDAKKEVEEAGGQAIIVQGDVTKEED
VVNLVQTAIKEFGTLDVMINNAGVENPVPSHELSLDNWNKVIDTNLTGAFLGSREAIKYFVENDIKGNVIN
MSSVHEMIPWPLFVHYAASKGGMKLMTETLALEYAPKGIRVNNIGPGAMNTPINAEKFADPVQRADVES
MIPMGYIGKPEEVAAVAAFLASSQASYVTGITLFADGGMTKYPSFQAGRG

The glucose dehydrogenase for co-factor regeneration mentioned herein preferably comprises an amino acid sequence having at least 80% identity to SEQ ID NO: 6, more preferably 85%, even more preferably 90%, even more preferably 95%, even more preferably 98%, even more preferably 99%, especially 100%. Particularly preferably, the inventive glucose dehydrogenase for co-factor regeneration comprises or consists of the amino acid sequence of SEQ ID NO: 6.

Alternatively, the glucose dehydrogenase for co-factor regeneration preferably comprises an amino acid sequence encoded by a nucleic acid having at least 80% identity to SEQ ID NO: 5, more preferably 85%, even more preferably 90%, even more preferably 95%, even more preferably 98%, even more preferably 99%, most preferably 100%. Particularly preferably, the nucleic acid encoding the inventive glucose dehydrogenase for co-factor regeneration comprises or consists of the nucleic acid sequence of SEQ ID NO: 5.

On aspect of the present invention relates to the use of a glucose dehydrogenase for co-factor regeneration, wherein the glucose dehydrogenase comprises or consists of an amino acid sequence selected from the group consisting of:
i) an amino acid sequence having at least 80% identity to SEQ ID NO: 6,
ii) an amino acid sequence encoded by a nucleic acid having at least 80% identity to SEQ ID NO: 5, and
iii) an amino acid sequence encoded by a nucleic acid that, under stringent conditions, binds to a nucleic acid molecule with the nucleic acid sequence of SEQ ID NO: 5.

The co-factor NAD(P)⁺, resulting during the reduction of D-psicose to allitol, is reduced to NAD(P)H by the use of formate and a formate dehydrogenase under the formation of CO₂ (co-factor regeneration).

Particularly preferably, a formate dehydrogenase comprising or consisting of the amino acid sequence of SEQ ID NO: 12 or a functional fragment of this formate dehydrogenase is used. The preferably used formate dehydrogenase is preferably encoded by the nucleic acid sequence of SEQ ID NO: 11. A “functional fragment” of the formate dehydrogenase comprises an N-terminally and/or C-terminally truncated variant of the formate dehydrogenase with the amino acid sequence of SEQ ID NO: 12, which has at least 50%, preferably at least 60%, more preferably at least 70%, even more preferably at least 80%, even more preferably at least 90%, even more preferably at least 95%, enzyme activity compared to a non-truncated formate dehydrogenase.

SEQ ID NO: 11:
ATGGCGAAAATACTTTGCGTTCTCTATGACGATCCGGTCGACGGCTACCCGAAGACCTATGCGCGCG
ACGACCTGCCGAAGATCGACCACTATCCGGGCGGGCAGACGCTGCCCACGCCCAAGGCGATCGACTT
CACGCCGGGCGCGCTGCTCGGCTCGGTCTCCGGCGAGCTCGGCCTGCGCAAATACCTGGAAGCCAAC
GGCCATACCTTCGTCGTCACCTCCGATAAGGACGGCCCGGATTCGGTGTTCGAGAGGGAACTCGTCG
ACGCCGACGTGGTGATCTCGCAGCCCTTCTGGCCGGCCTATCTGACGCCCGAGCGCATCGCCAAGGC
GAAGAACCTGAAGCTCGCGCTCACCGCCGGCATCGGCTCCGATCATGTCGATCTTCAGTCAGCTATCG
ACCGTGGCATCACTGTGGCCGAAGTCACATATTGCAACTCGATCAGCGTCGCCGAGCACGTGGTGAT
GATGATCCTCGGCCTGGTACGAAACTACATTCCCTCGCATGACTGGGCGCGCAAGGGCGGCTGGAAC
ATAGCCGACTGCGTAGAGCACTCCTACGACCTCGAGGGCATGACCGTCGGCTCGGTGGCCGCCGGCC
GCATCGGCCTCGCCGTGCTGCGCCGCCTCGCGCCGTTCGACGTGAAGCTGCACTATACCGACCGCCA
CCGTCTGCCAGAAGCGGTCGAGAAGGAGCTGGGCCTCGTCTGGCACGATACCCGCGAGGACATGTA
CCCGCATTGCGACGTGGTCACGCTCAACGTGCCGCTGCACCCCGAAACCGAGCACATGATCAATGAC
GAGACGCTGAAGCTGTTCAAGCGCGGCGCCTATATCGTCAACACCGCCCGCGGCAAGCTCGCCGACC
GCGACGCCATCGTCCGCGCGATCGAGAGCGGGCAGCTCGCGGGCTATGCCGGCGACGTGTGGTTCC
CGCAGCCGGCTCCGAAGGACCACCCCTGGCGCACCATGAAGTGGGAAGGCATGACGCCGCACATCT
CCGGCACCTCGCTCTCTGCCCAGGCGCGCTACGCGGCGGGCACGCGCGAGATCCTCGAATGCTTCTT
CGAGGGCCGGCCGATCCGCGACGAGTACCTGATCGTGCAGGGCGGCGCGCTCGCCGGCACCGGCGC
GCATTCCTACTCGAAGGGCAATGCGACCGGCGGTTCGGAAGAGGCCGCGAAGTTCAAGAAGGCTGG
CTGA
SEQ ID NO: 12:
MAKILCVLYDDPVDGYPKTYARDDLPKIDHYPGGQTLPTPKAIDFTPGALLGSVSGELGLRKYLEANGHTFV
VTSDKDGPDSVFERELVDADVVISQPFWPAYLTPERIAKAKNLKLALTAGIGSDHVDLQSAIDRGITVAEVT
YCNSISVAEHVVMMILGLVRNYIPSHDWARKGGWNIADCVEHSYDLEGMTVGSVAAGRIGLAVLRRLAP
FDVKLHYTDRHRLPEAVEKELGLVWHDTREDMYPHCDVVTLNVPLHPETEHMINDETLKLFKRGAYIVNT
ARGKLADRDAIVRAIESGQLAGYAGDVWFPQPAPKDHPWRTMKWEGMTPHISGTSLSAQARYAAGTR
EILECFFEGRPIRDEYLIVQGGALAGTGAHSYSKGNATGGSEEAAKFKKAG

According to a further preferred embodiment of the present invention, the formate dehydrogenase used for co-factor regeneration comprises an amino acid sequence is selected from the group consisting of:
i) an amino acid sequence having at least 80% identity to SEQ ID NO: 12,
ii) an amino acid sequence encoded by a nucleic acid having at least 80% identity to SEQ ID NO: 11, and
iii) an amino acid sequence encoded by a nucleic acid that, under stringent conditions, binds to a nucleic acid molecule with the nucleic acid sequence of SEQ ID NO: 11, or a functional fragment thereof.

The formate dehydrogenase preferably comprises an amino acid sequence having at least 80% identity to SEQ ID NO: 12, more preferably 85%, even more preferably 90%, even more preferably 95%, even more preferably 98%, even more preferably 99%, especially 100%.

Alternatively, the formate dehydrogenase preferably comprises an amino acid sequence encoded by a nucleic acid having at least 80% identity to SEQ ID NO: 11, more preferably 85%, even more preferably 90%, even more preferably 95%, even more preferably 98%, even more preferably 99%, especially 100%.

A further aspect of the present invention relates to the use of a formate dehydrogenase for co-factor regeneration or of a functional fragment thereof, the formate dehydrogenase comprising or consisting of an amino acid sequence selected from the group consisting of:
i) an amino acid sequence having at least 80% identity to SEQ ID NO: 12,
ii) an amino acid sequence encoded by a nucleic acid having at least 80% identity to SEQ ID NO: 11, and
iii) an amino acid sequence encoded by a nucleic acid that, under stringent conditions, binds to a nucleic acid molecule with the nucleic acid sequence of SEQ ID NO: 11.

The enzymatic strategy presented herein, in combination with the co-factor regeneration, allows for a biocatalytic, environmentally friendly, and highly efficient production process for the preparation of allitol.

### Materials

D-Psicose was obtained from TCI and Hunan Garden Naturals Inc. (China), allitol was obtained from TCI, D-fructose, lysozyme and methanol were obtained from PanReac AppliChem (ITW Reagents), D-glucose, sodium gluconate, IPTG (isopropyl-β-D-thiogalactopyranoside) were obtained from Sigma-Aldrich, potassium dihydrogen phosphate, di-potassium hydrogen phosphate, NAD⁺, NADH disodium salt, and sodium dodecyl sulfate (SDS) were obtained from Carl Roth and triethanolamine (TEA) was obtained from Chem-Lab NV.

### Production of the Enzymes & Preparation of the Lysates

General Information about the Expression of Recombinant Enzymes in E. coli

For recombinant enzyme production in an Escherichia coli strain, the gene to be expressed was first amplified using the genomic DNA or its equivalent synthetically adapted to the codon use of E. coli as template together with the specific oligonucleotides, which additionally carried recognition sequences for restriction endonucleases, and isolated from the reaction mixture. After nucleic acid digestion with the restriction enzymes SphI and HindIII, the gene fragment encoding the target enzyme was ligated into the SphI- and HindIII-cleaved backbone of the expression vector pQE70-Kan. The ligation product was transformed into chemically competent E. coli Top10F cells, and the resulting colonies were used for plasmid isolation and restriction analysis.

The result of the cloning step was verified by restriction enzyme digestion and DNA sequencing. The resulting construct carries the target gene under the IPTG-inducible T5 promotor.

For overexpression of the enzyme in E. coli, the resulting expression plasmid was transformed into the competent expression cells RB791. After 24 h of incubation at 37° C., the resulting colonies were inoculated for the expression tests in LB medium.

The next day, expression cultures were inoculated therewith at an optical density OD₅₅₀ of 0.02 and shaken at 37° C. until an OD₅₅₀ of 0.3 was reached. Subsequently, the temperature was lowered to 25° C., and the cultures were induced with 0.1 mM of IPTG when an OD₅₅₀ of 0.5 was reached. After 22 h, the cultures were harvested (separated from the medium by centrifugation in the form of a cell pellet) and analyzed for the expression of the recombinant enzyme by means of SDS gel electrophoresis and activity determination (application in use test or optical enzymatic assay).

### Preparation of Cell Lysates by Means of Sonifier Disruption

To prepare a cell suspension, the cell pellet prepared according to the above method was weighed into a suitable container, mixed with a buffer and lysozyme (final concentration 0.5 mg/ml) (e.g., triethanolamine (TEA)-HCl) and dissolved under stirring. The mass fraction of biomass is usually 20%, the remainder is buffer.

A Branson Sonifier 450 was used for cell disruption. The suspension was treated three times with 15 ultrasonic pulses each (device settings: Timer=15; Duty Cycle=50; Output Control=3-5).

The resulting homogenate was centrifuged for 10 min at 4° C. and 16000 rpm (Eppendorf Centrifuge 5417R) to separate the insoluble cell fragments and obtain the lysate.

TABLE 1
Enzyme classes and donor organisms for the enzymes used in the examples (SDR
= oxidoreductase from the short-chain dehydrogenase/reductase family).

| Enzyme type | Catalyzed | | |
|---|---|---|---|
| (EC class) | reaction | Donor organism | Literature |
| D-Psicose-3-epimerase | D-fructose [symbol] | Clostridium | (Mu et al., 2011; Chan |
| (EC 5.1.3.30) | D-psicose | cellulolyticum H10 | et al., 2012) |
| SDR I | D-psicose [symbol] | Gluconobacter | (NCBI Protein Database: |
| | allitol | frateurii | WP_063903495.1); |
| | | (DSM 7146) | SEQ ID NO: 2 |
| SDR II | D-psicose [symbol] | Kozakia baliensis | (NCBI Protein Database: |
| | allitol | | WP_070401870.1); |
| | | | SEQ ID NO: 8 |
| SDR III | D-psicose [symbol] | Providencia | (NCBI Protein Database: |
| | allitol | heimbachae | WP_068907433.1); |
| | | | SEQ ID NO: 10 |
| Alcohol dehydrogenase | 2-propanol [symbol] | (Geo-)Bacillus | (Sakoda & Imanaka, |
| (ADH) | acetone | stearothermophilus | 1992; NCBI Protein |
| | | NCA1503 | Database: |
| | | | WP_033015595.1); |
| | | | SEQ ID NO: 4 |
| Glucose dehydrogenase | D-glucose [symbol] | Priestia | (NCBI Protein Database: |
| (GDH) | D-gluconate (via | megaterium | MDQ0804260.1); SEQ |
| | D-gluconolactone) | | ID NO: 6 |
| Formate dehydrogenase | formate → CO₂ | Starkeya | (NCBI Protein Database: |
| (FDH; EC 1.17.1.9) | | novella/Ancylobacter | WP_013168047.1); SEQ |
| | | novellus | ID NO: 12 |

### Analytical Methods

### High Performance Liquid Chromatography

An Agilent HPLC 1260 Infinity II Series system was used to quantify D-psicose, D-fructose, D-glucose, and allitol by HPLC (High Performance Liquid Chromatography). Detection was carried out using a refractive index detector (RI detection). For the measurement, a Phenomenex Rezex RPM-Monosaccharide Pb+2 (8%) column with an appropriate pre-column was used and eluted isocratically with ultrapure water.

### High Performance Anion Exchange Chromatography

A Dionex ICS6000 system with an AS-AP Autosampler was used to quantify D-gluconic acid/D-gluconate by means of HPAEC (High Performance Anion Exchange Chromatography). The measurement was performed by means of conductivity detection (CD) coupled to a Dionex AERS 500 electrolytically regenerated suppressor in external water mode. A Dionex IonPac AS11-HC-4 μm column with an appropriate pre-column and a NaOH gradient was used to separate the analytes. The solvent was additionally pretreated with a Dionex ATC Anion Trap column.

### Determination of Enzyme Activities (Optical-Enzymatic Assay)

Enzyme activities in the lysates were determined using a Shimadzu UV-1900 spectrophotometer. For this purpose, the formation or consumption of NAD(P)H was monitored at a wavelength of 340 nm via the change of absorption. The measurements were performed with 0.2 mM of co-factor (NAD(P)⁺ or NAD(P)H). For this purpose, 20 μl of a 10 mM stock solution of the co-factor were plated in a cuvette (Greiner bio-one Semi-Micro-Cuvette made of polystyrene), and the desired pH was adjusted with 100 mM of TEA-HCl buffer (870 μl). 10 μl of lysate (diluted or undiluted) and 100 μl of substrate solution were added to the cuvette and measurement was started immediately. The measurements were carried out at 25° C. by default. The enzyme activity of the lysate can be determined in U/ml (based on the volume of the lysate) or U/g (based on the biomass used for production) using the extinction coefficient of NADH/NADPH at 340 nm (ε=6220 l mol⁻¹ cm⁻¹). Here, 1 U stands for 1 μmol substrate conversion per minute (1 U=1 μmol/min=1.67·10⁻⁸ kat).

The following examples describe preferred variants of the method according to the invention in more detail. The lysates used in these examples were prepared according to the procedures described above.

### Example 1

### Conversion of D-Fructose to Allitol—Comparison of Regeneration Via ADH and FDH

The following components were mixed in two 2 ml glass vials (Vial 1=ADH regeneration and 2=FDH regeneration): 100 μl of a 1 M TEA-HCl buffer (pH 8), 150 μl of a D-fructose solution (500 g/l), 35 μl of D-psicose-3-epimerase lysate, 50 μl of SDR I lysate and 10 μl of a 10 mM NAD⁺ solution. 55.5 μl of ADH lysate, 50 μl of 2-propanol, and 49.5 μl of H₂O were added to vial 1 and 55.5 μl of FDH lysate, 60 μl of an 8 M sodium formate solution, and 39.5 μl of H₂O were added to vial 2.

The preparations were incubated under continuous shaking (Eppendorf Thermomixer, 35° C., 800 rpm) for a total of 24 h.

For analysis, 100 μl of the preparation was treated with 200 μl of methanol and incubated in the Eppendorf Thermomixer at 60° C. and 1200 rpm for 15 min. The sample was briefly centrifuged in a centrifuge, treated with 700 μl of deionized water, vortexed, and then centrifuged for 5 min at max. g. 200 μl of the supernatant were transferred to a HPLC vial with an insert and measured by HPLC (RI detection).

In this manner, 72.7% of the D-fructose in vial 1 (ADH regeneration) and 11.6% of the D-fructose in vial 2 (FDH regeneration) could be converted to allitol.

This example demonstrates that co-factor regeneration with ADH at high substrate concentrations (150 g/l) allow higher conversions than co-factor regeneration with FDH.

### Example 2

### Conversion of D-Fructose to Allitol—Co-Factor Regeneration by GDH and D-Glucose

The reactions were carried out in a Multifors tabletop bioreactor (Infors AG). A glass reactor (volume 11) with an agitator and a pH electrode was used as vessel. The pH was controlled by adding 5M NaOH or 1M H₂SO₄.

At the start, 52.5 g of D-fructose, 52.5 g of D-glucose, 172.5 ml of deionized water, and 26.5 ml of a 500 mM KPP buffer (pH 7.5) were placed in the reactor and brought to 35° C. while stirring.

To start the reaction, 17.5 ml of D-psicose-3-epimerase lysate were added. Then, 24.5 ml of SDR I lysate, 2.2 kU of GDH lysate and 3.5 ml of a 10 mM NAD⁺ solution were added.

For analysis, 50 μl of the preparation were treated with 200 μl of methanol and incubated in the Eppendorf Thermomixer at 60° C. and 1200 rpm for 15 min. The sample was briefly centrifuged in a centrifuge, treated with 750 μl of deionized water, vortexed, and then centrifuged for 5 min at max. g. 200 μl of the supernatant were transferred to a HPLC vial with an insert and measured by HPLC (RI detection). For the HPAEC measurements (conductivity detection), the clear supernatant was diluted 1:250.

In this way, 90.6% of the D-fructose (150 g/l) was converted to allitol within 30 h (found concentration: 119 g/l), and the D-glucose (150 g/l) was completely converted to D-gluconate (found concentration: 167 g/l).

The reactor content was stirred for 1 h at 70° C. The hot reaction mixture was filtered through a glass frit (P4, 10-16 μm) by applying vacuum. The filtrate was treated with acetone (final volume fraction 50%) and stored in the refrigerator overnight at 4° C. The precipitate was filtered off (glass frit P4, 10-16 μm) and washed with ice-cold acetone. The product was dried in a vacuum drying cabinet for 24 h at 50° C. An HPLC and HPAEC analysis of the product showed that allitol could be obtained with high purity (≥97%) from the D-gluconate-containing solution.

This example demonstrates that an efficient conversion of D-fructose (150 g/l) to allitol is possible in a 15-fold substrate concentration compared to Takeshita et al. (2000).

### REFERENCES

Hough, L, & Stacey, B. E. (1963). The occurrence of D-ribohexulose in Itea ilicifolia, Itea virginica, and Itea yunnanensis. Phytochemistry, 2(4), 315-320. https://doi.org/10.1016/S0031-9422(00)84854-2
Izumori, K. (2006). Izumoring: a strategy for bioproduction of all hexoses. Journal of Biotechnology, 124(4), 717-722. https://doi.org/10.1016/j.jbiotec.2006.04.016
Hassanin, H. A. M., Mu, W., Koko, M. Y. F., Zhang, T., Masamba, K., & Jiang, B. (2017). Allitol: production, properties and applications. International Journal of Food Science and Technology, 52(1), 91-97. https://doi.org/10.1111/ijfs.13290
Gullapalli, P., Takata, G., Poonperm, W., Rao, D., Morimoto, K., Akimitsu, K., Tajima, S., & Izumori, K. (2007). Bioproduction of D-psicose from allitol with Enterobacter aerogenes IK7: a new frontier in rare ketose production. Bioscience, Biotechnology, and Biochemistry, 71(12), 3048-3054. https://doi.org/10.1271/bbb.70450
Poonperm, W., Takata, G., Ando, Y., Sahachaisaree, V., Lumyong, P., Lumyong, S., & Izumori, K. (2007). Efficient conversion of allitol to D-psicose by Bacillus pallidus Y25. Journal of Bioscience and Bioengineering, 103(3), 282-285. https://doi.org/10.1263/jbb.103.282
Takeshita, K., Shimonishi, T., & Izumori, K. (1996). Production of L-psicose from allitol by Gluconobacter frateurii IFO 3254. Journal of Fermentation and Bioengineering, 81(3), 212-215. https://doi.org/10.1016/0922-338X(96)82210-0
Izumori, K., Khan, A. R., Okaya, H., & Tsumura, T. (1993). A New Enzyme, D-Ketohexose 3-Epimerase, from Pseudomonas sp. ST-24. Bioscience, Biotechnology, and Biochemistry, 57(6), 1037-1039. https://doi.org/10.1271/bbb.57.1037
Zhang, W., Yu, S., Zhang, T., Jiang, B., & Mu, W. (2016). Recent advances in D-allulose: Physiological functionalities, applications, and biological production. Trends in Food Science and Technology, 54, 127-137. https://doi.org/10.1016/j.tifs.2016.06.004
Jiang, S., Xiao, W., Zhu, X., Yang, P., Zheng, Z., Lu, S., Jiang, S., Zhang, G., & Liu, J. (2020). Review on D-Allulose: In vivo Metabolism, Catalytic Mechanism, Engineering Strain Construction, Bio-Production Technology. Frontiers in Bioengineering and Biotechnology, 8, 26. https://doi.org/10.3389/fbioe.2020.00026
Muniruzzaman, S., Tokunaga, H., & Izumori, K. (1995). Conversion of D-psicose to allitol by Enterobacter agglomerans strain 221e. Journal of Fermentation and Bioengineering, 79(4), 323-327. https://doi.org/10.1016/0922-338X(95)93989-W
Han, W., Zhu, Y., Men, Y., Yang, J., Uu, C., & Sun, Y. (2014). Production of allitol from D-psicose by a novel isolated strain of Klebsiella oxytoca G4A4. Journal of Basic Microbiology, 54(10), 1073-1079. https://doi.org/10.1002/jobm.201300647
Zhu, Y., Li, H., Uu, P., Yang, J., Zhang, X., & Sun, Y. (2015). Construction of allitol synthesis pathway by multi-enzyme coexpression in Escherichia coli and its application in allitol production. Journal of Industrial Microbiology & Biotechnology, 42(5), 661-669. https://doi.org/10.1007/s10295-014-1578-1
Wen, X., Un, H., Ren, Y., U, C., Zhang, C., Un, J., & Un, J. (2022). Allitol bioproduction by recombinant Escherichia coli with NADH regeneration system co-expressing ribitol dehydrogenase (RDH) and formate dehydrogenase (FDH) in individual or in fusion. Electronic Journal of Biotechnology, 55, 91-98. https://doi.org/10.1016/j.ejbt.2021.11.007
Wang, L, Chen, K., Zheng, P., Huo, X., Uao, F., Zhu, L, Hu, M., & Tao, Y. (2023). Enhanced production of D-psicose from D-fructose by a redox-driven multi-enzyme cascade system. Enzyme and Microbial Technology, 163, 110172. https://doi.org/10.1016/j.enzmictec.2022.110172
Zhao, J., Guo, Y., Li, Q., Chen, J., Niu, D., & Liu, J. (2022). Reconstruction of a Co-factor Self-Sufficient Whole-Cell Biocatalyst System for Efficient Biosynthesis of Allitol from D-Glucose. Journal of Agricultural and Food Chemistry, 70(12), 3775-3784. https://doi.org/10.1021/acs.jafc.2c00440
Feng, T., Wang, Z., Li, H., Li, Q., Guo, Y., Zhao, J., & Liu, J. (2023). Whole-cell biotransformation for simultaneous synthesis of allitol and D-gluconic acid in recombinant Escherichia coli. Journal of Bioscience and Bioengineering, 135(6), 433-439. https://doi.org/10.1016/j.jbiosc.2023.03.004
Hassanin, H. A. M., Letsididi, R., Koko, M. Y. F., Mu, W., Elferga, A., & Jiang, B. (2016). Synthesis of allitol from D-psicose using ribitol dehydrogenase and formate dehydrogenase. Tropical Journal of Pharmaceutical Research, 15(12), 2701-2708. https://doi.org/10.4314/tjpr.v15i12.23
Xu, J., Zhou, H., Yu, H., Deng, T., Wang, Z., Zhang, H., Wu, J., & Yang, L (2021). Computational design of highly stable and soluble alcohol dehydrogenase for NADPH regeneration. Bioresources and Bioprocessing, 8, 12. https://doi.org/10.1186/s40643-021-00362-w
Takeshita, K., Ishida, Y., Takada, G., & Izumori, K. (2000). Direct Production of Allitol from D-Fructose by a Coupling Reaction Using D-Tagatose 3-Epimerase, Ribitol Dehydrogenase and Formate Dehydrogenase. Journal of Bioscience and Bioengineering, 90(5), 545-548. https://doi.org/10.1016/S1389-1723(01)80038-4
Al-Rabiah, A. A., Boz, I., Akhmedov, V. M., Mostafa, M. M. M., & Bagabas, A. A. (2022). Highly Selective Gas-Phase Catalytic Hydrogenation of Acetone to Isopropyl Alcohol. Catalysts, 12(10), 1251. https://doi.org/10.3390/catal12101251
Kornecki, J. F., Carballares, D., Tardioli, P. W., Rodrigues, R. C., Berenguer-Murcia, A., Alcántara, A. R., & Fernandez-Lafuente, R. (2020). Enzyme production of D-gluconic acid and glucose oxidase: successful tales of cascade reactions. Catalysis Science & Technology, 10(17), 5740-5771. https://doi.org/10.1039/D0CY00819B
Lin, B., & Tao, Y. (2017). Whole-cell biocatalysts by design. Microbial Cell Factories, 16, 106. https://doi.org/10.1186/s12934-017-0724-7
Altschul, S. F., Gish, W., Miller, W., Myers, E. W., & Lipman, D. J. (1990). Basic local alignment search tool. Journal of Molecular Biology, 215(3), 403-410. https://doi.org/10.1016/S0022-2836(05)80360-2
Henikoff, S., & Henikoff, J. G. (1992). Amino acid substitution matrices from protein blocks. Proceedings of the National Academy of Sciences of the United States of America, 89(22), 10915-10919. https://doi.org/10.1073/pnas.89.22.10915
Sambrook, J., Fritsch, E. R., & Maniatis, T. (1989). Molecular Cloning: A Laboratory Manual (2nd ed.). Cold Spring Harbor, NY: Cold Spring Harbor Laboratory Press.
Mu, W., Chu, F., Xing, Q., Yu, S., Zhou, L, & Jiang, B. (2011). Cloning, Expression, and Characterization of a D-Psicose 3-Epimerase from Clostridium cellulolyticum H10. Journal of Agricultural and Food Chemistry, 59(14), 7785-7792. https://doi.org/10.1021/jf201356q
Chan, H.-C., Zhu, Y., Hu, Y., Ko, T.-P., Huang, C.-H., Ren, F., Chen, C.-C., Ma, Y., Guo, R.-T., & Sun, Y. (2012). Crystal structures of D-psicose 3-epimerase from Clostridium cellulolyticum H10 and its complex with ketohexose sugars. Protein & Cell, 3(2), 123-131. https://doi.org/10.1007/s13238-012-2026-5
Protein [Internet]. Bethesda (MD): National Library of Medicine (US), National Center for Biotechnology Information. Accession No. WP_063903495.1, SDR family oxidoreductase [Gluconobacter frateurii]. Available at: https://www.ncbi.nlm.nih.gov/protein/WP_063903495.1 (Accessed on 8 Apr. 2024)
Protein [Internet]. Bethesda (MD): National Library of Medicine (US), National Center for Biotechnology Information. Accession No. WP_070401870.1, SDR family oxidoreductase [Kozakia baliensis]. Available at: https://www.ncbi.nlm.nih.gov/protein/WP_070401870.1/ (Accessed on 8 Apr. 2024)
Protein [Internet]. Bethesda (MD): National Library of Medicine (US), National Center for Biotechnology Information. Accession No. WP_068907433.1, SDR family oxidoreductase [Providencia heimbachae]. Available at: https://www.ncbi.nlm.nih.gov/protein/WP_068907433.1/ (Accessed on 8 Apr. 2024)
Sakoda, H., & Imanaka, T. (1992). Cloning and sequencing of the gene coding for alcohol dehydrogenase of Bacillus stearothermophilus and rational shift of the optimum pH. Journal of Bacteriology, 174(4), 1397-1402. https://doi.org/10.1128/jb.174.4.1397-1402.1992
Protein [Internet]. Bethesda (MD): National Library of Medicine (US), National Center for Biotechnology Information. Accession No. WP_033015595.1, MULTISPECIES: alcohol dehydrogenase AdhP [Geobacillus]. Available at: https://www.ncbi.nlm.nih.gov/protein/WP_033015595.1/(Accessed on 8 Apr. 2024)
Protein [Internet]. Bethesda (MD): National Library of Medicine (US), National Center for Biotechnology Information. Accession No. MDQ0804260.1, Glucose 1-dehydrogenase [Priestia megaterium]. Available at: https://www.ncbi.nlm.nih.gov/protein/MDQ0804260.1 (Accessed on 8 Apr. 2024)
Protein [Internet]. Bethesda (MD): National Library of Medicine (US), National Center for Biotechnology Information. Accession No. WP_013168047.1, NAD-dependent formate dehydrogenase [Ancylobacter novellus]. Available at: https://www.ncbi.nlm.nih.gov/protein/WP_013168047.1 (Accessed on 8 Apr. 2024)

1. A method for producing allitol in vitro, the method comprising:
treating D-fructose present in an aqueous solution with an epimerase to form D-psicose; and
treating D-psicose with an NAD(P)H-dependent oxidoreductase to produce allitol, and enzymatically reducing the resulting NAD(P)⁺ to NAD(P)H with a hydrogen donor,
wherein the hydrogen donor is a secondary alcohol.

2. The method of claim 1, wherein the secondary alcohol is 2-propanol.

3. The method of claim 1, wherein the secondary alcohol is D-glucose.

4. The method of claim 1, wherein the step of treating D-fructose and the step of treating D-psicose is carried out as a one-pot reaction without isolation of the D-psicose.

5. The method of claim 1, wherein the epimerase and the NAD(P)H-dependent oxidoreductase are present as lysates of cells expressing the epimerase and the NAD(P)H-dependent oxidoreductase.

6. The method of claim 1, wherein the NAD(P)H-dependent oxidoreductase in the step of treating the D-psicose comprises an amino acid sequence selected from the group consisting of:
i) an amino acid sequence having at least 80% identity to SEQ ID NO: 2, SEQ ID NO: 8, or SEQ ID NO: 10,
ii) an amino acid sequence encoded by a nucleic acid having at least 80% identity to SEQ ID NO: 1, SEQ ID NO: 7, or SEQ ID NO: 9, and
iii) an amino acid sequence encoded by a nucleic acid that, under stringent conditions, binds to a nucleic acid molecule with the nucleic acid sequence of SEQ ID NO: 1, SEQ ID NO: 7, or SEQ ID NO: 9.

7. The method of claim 1, further comprising using an alcohol dehydrogenase to catalyze the reduction of NAD(P)⁺ during the step of treating D-psicose.

8. The method of claim 1, further comprising using a glucose dehydrogenase to catalyze the reduction of NAD(P)⁺ during the step of treating D-psicose.

9. (canceled)

10. The method of claim 1, further comprising using a formate dehydrogenase in the step of treating D-psicose, wherein the formate dehydrogenase comprise an amino acid sequence selected from the group consisting of:
i) an amino acid sequence having at least 80% identity to SEQ ID NO: 12,
ii) an amino acid sequence encoded by a nucleic acid having at least 80% identity to SEQ ID NO: 11, and
iii) an amino acid sequence encoded by a nucleic acid that, under stringent conditions, binds to a nucleic acid molecule with the nucleic acid sequence of SEQ ID NO: 11.

11. The method of claim 7, wherein the alcohol dehydrogenase comprises an amino acid sequence selected from the group consisting of:
i) an amino acid sequence having at least 80% identity to SEQ ID NO: 4,
ii) an amino acid sequence encoded by a nucleic acid having at least 80% identity to SEQ ID NO: 3, and
iii) an amino acid sequence encoded by a nucleic acid that, under stringent conditions, binds to a nucleic acid molecule with the nucleic acid sequence of SEQ ID NO: 3.

12. The method of claim 8, wherein the glucose dehydrogenase comprises an amino acid sequence selected from the group consisting of:
i) an amino acid sequence having at least 80% identity to SEQ ID NO: 6,
ii) an amino acid sequence encoded by a nucleic acid having at least 80% identity to SEQ ID NO: 5, and
iii) an amino acid sequence encoded by a nucleic acid that, under stringent conditions, binds to a nucleic acid molecule with the nucleic acid sequence of SEQ ID NO: 5.

13. A method for producing allitol from D-psicose in vitro, the method comprising:
treating D-psicose present in an aqueous solution with an oxidoreductase to reduce D-psicose to allitol,
wherein the oxidoreductase comprises an amino acid sequence selected from the group consisting of:
i) an amino acid sequence having at least 80% identity to SEQ ID NO: 2, SEQ ID NO: 8, or SEQ ID NO: 10,
ii) an amino acid sequence encoded by a nucleic acid having at least 80% identity to SEQ ID NO: 1, SEQ ID NO: 7, or SEQ ID NO: 9, and
iii) an amino acid sequence encoded by a nucleic acid that, under stringent conditions, binds to a nucleic acid molecule with the nucleic acid sequence of SEQ ID NO: 1, SEQ ID NO: 7, or SEQ ID NO: 9.

14. A method for regenerating a co-factor or a functional fragment thereof using a formate dehydrogenase, wherein the formate dehydrogenase comprises an amino acid sequence selected from the group consisting of:
i) an amino acid sequence having at least 80% identity to SEQ ID NO: 12,
ii) an amino acid sequence encoded by a nucleic acid having at least 80% identity to SEQ ID NO: 11, and
iii) an amino acid sequence encoded by a nucleic acid that, under stringent conditions, binds to a nucleic acid molecule with the nucleic acid sequence of SEQ ID NO: 11.

15. The method of claim 14, wherein the formate dehydrogenase consists of an amino acid sequence selected from the group consisting of:
i) an amino acid sequence having at least 80% identity to SEQ ID NO: 12,
ii) an amino acid sequence encoded by a nucleic acid having at least 80% identity to SEQ ID NO: 11, and
iii) an amino acid sequence encoded by a nucleic acid that, under stringent conditions, binds to a nucleic acid molecule with the nucleic acid sequence of SEQ ID NO: 11.


SEQUENCE LISTING (12 sequences)

SEQ ID NO: 1 - DNA, 785 bp, Gluconobacter frateurii
  source 1..785: mol_type = genomic DNA
atgacacagtctcttcagggcaagatcgtcgccattactggcgcggcttcgggcattggcctcgaatgcgcccgctatctcatcgaagctggcgcggtggtctatcttctggaccgtgacgccaaaactctcgaagacaaaacggcagaactcggcagccaggcccatgcgatcatcgtcgatctcttcgactacaaaaccgtagatgctgcggtcgcgcagatcgttgaggagcagggccggatcgatgttttccacgccaacgccggcgcgtatgtgggcggcaatgtctgggaaggggatcccgatagctgggatgcgatgctgcacctgaacatcaatgcagctttccgctcggtccgtgccgtgctgccgcagatgatgaagcaggaaagtggcgatatcgtcatgaccagctcgatcgcgggcatgatccccatcatggccgagccgatctacacggcctcgaaacatgccgtgcaggcattcgtacatacggtgcgccgacaggtcgcgaagtatggaattcgtgtgggtgcaatccagcctggtcctgtagtcacgcctttgctgaaagactgggatcaggcccgtcttgaagccaacatcaaagcgggtgccctgatggaagccaaggaagtcgccgaagccctgatcttcatcctgacacgttcaaagggtgtcatggtgcgggatctggtcgttctgccacataacttcgacgcctaagcttaagcggccgcactcgagcaccaccaccaccaccactgagatccggctgctaa

SEQ ID NO: 2 - protein, 242 aa, Gluconobacter frateurii
MTQSLQGKIVAITGAASGIGLECARYLIEAGAVVYLLDRDAKTLEDKTAELGSQAHAIIVDLFDYKTVDAAVAQIVEEQGRIDVFHANAGAYVGGNVWEGDPDSWDAMLHLNINAAFRSVRAVLPQMMKQESGDIVMTSSIAGMIPIMAEPIYTASKHAVQAFVHTVRRQVAKYGIRVGAIQPGPVVTPLLKDWDQARLEANIKAGALMEAKEVAEALIFILTRSKGVMVRDLVVLPHNFDA

SEQ ID NO: 3 - DNA, 1014 bp, Geobacillus stearothermophilus
  source 1..1014: mol_type = genomic DNA
atgaaagctgcagttgtggaacaatttaaaaagccgttacaagtgaaagaagtggaaaaacctaagatctcatacggggaagtattagtgcgcatcaaagcgtgtggggtatgccatacagacttgcatgccgcacatggcgactggcctgtaaagcctaaactgcctctcattcctggccatgaaggcgtcggtgtaattgaagaagtaggtcctggggtaacacatttaaaagttggagatcgcgtaggtatcccttggctttattcggcgtgcggtcattgtgactattgcttaagcggacaagaaacattatgcgaacgtcaacaaaacgctggctattccgtcgatggtggttatgctgaatattgccgtgctgcagccgattatgtcgtaaaaattcctgataacttatcgtttgaagaagccgctccaatcttttgcgctggtgtaacaacatataaagcgctcaaagtaacaggcgcaaaaccaggtgaatgggtagccatttacggtatcggcgggcttggacatgtcgcagtccaatacgcaaaggcgatggggttaaacgtcgttgctgtcgatttaggtgatgaaaaacttgagcttgctaaacaacttggtgcagatcttgtcgtcaatccgaaacatgatgatgcagcacaatggataaaagaaaaagtgggcggtgtgcatgcgactgtcgtcacagctgtttcaaaagccgcgttcgaatcagcctacaaatccattcgtcgcggtggtgcttgcgtactcgtcggattaccgccggaagaaatacctattccaattttcgatacagtattaaatggagtaaaaattattggttctatcgttggtacgcgcaaagacttacaagaggcacttcaatttgcagcagaaggaaaagtaaaaacaattgtcgaagtgcaaccgcttgaaaacattaacgacgtattcgatcgtatgttaaaagggcaaattaacggccgcgtcgtgttaaaagtagattaa

SEQ ID NO: 4 - protein, 337 aa, Geobacillus stearothermophilus
MKAAVVEQFKKPLQVKEVEKPKISYGEVLVRIKACGVCHTDLHAAHGDWPVKPKLPLIPGHEGVGVIEEVGPGVTHLKVGDRVGIPWLYSACGHCDYCLSGQETLCERQQNAGYSVDGGYAEYCRAAADYVVKIPDNLSFEEAAPIFCAGVTTYKALKVTGAKPGEWVAIYGIGGLGHVAVQYAKAMGLNVVAVDLGDEKLELAKQLGADLVVNPKHDDAAQWIKEKVGGVHATVVTAVSKAAFESAYKSIRRGGACVLVGLPPEEIPIPIFDTVLNGVKIIGSIVGTRKDLQEALQFAAEGKVKTIVEVQPLENINDVFDRMLKGQINGRVVLKVD

SEQ ID NO: 5 - DNA, 786 bp, Priestia megaterium
  source 1..786: mol_type = genomic DNA
atgtatacagatttaaaagataaagtagttgtaattacaggtggatcaacaggtttaggacgcgcaatggctgttcgtttcggtcaagaagaagcaaaagttgttattaactattacaacaatgaagaagaagctttagatgcgaaaaaagaagtagaagaagcaggcggacaagcaatcatcgttcaaggcgacgtaacaaaagaagaagatgttgtaaaccttgttcaaacagctattaaagaattcggtacattagacgttatgattaataacgctggtgttgaaaacccagttccttctcatgagttatctttagacaactggaataaagttattgatacaaacttaacaggtgcattcttaggaagccgtgaagcaatcaaatattttgttgaaaacgacattaaaggaaacgttattaacatgtctagtgttcatgaaatgattccttggccattatttgttcattacgcagcaagtaaaggcggtatgaaactaatgacggaaacattggctcttgaatatgcgccaaaaggtatccgcgtaaataacattggaccaggtgcgatgaacacaccaattaacgcagagaaatttgcagatcctgtacaacgtgcagacgtagaaagcatgattccaatgggttacatcggtaaaccagaagaagtagcagcagttgcagcattcttagcatcatcacaagcaagctatgtaacaggtattacattatttgctgatggtggtatgacgaaatacccttctttccaagcaggaagaggctaa

SEQ ID NO: 6 - protein, 261 aa, Priestia megaterium
MYTDLKDKVVVITGGSTGLGRAMAVRFGQEEAKVVINYYNNEEEALDAKKEVEEAGGQAIIVQGDVTKEEDVVNLVQTAIKEFGTLDVMINNAGVENPVPSHELSLDNWNKVIDTNLTGAFLGSREAIKYFVENDIKGNVINMSSVHEMIPWPLFVHYAASKGGMKLMTETLALEYAPKGIRVNNIGPGAMNTPINAEKFADPVQRADVESMIPMGYIGKPEEVAAVAAFLASSQASYVTGITLFADGGMTKYPSFQAGRG

SEQ ID NO: 7 - DNA, 729 bp, Kozakia baliensis
  source 1..729: mol_type = genomic DNA
atgacctctcctctccagggtaagatagccgccatcacgggcggggcttcgggcatcggcctcgaatgtgtccgccagatcgccgcaagtggtgccacggtttatattctcgaccgcgaccatcaggcgctcgacaaggcgcgcgaagaattgggcgagcgcgttcataccatcgaggtcgatctcttccgttacgaaacggtcgatcgcgccatcgaaaccatcgtgtccgaacaaggacgcatcgacattctccatgtcaatgcgggcgcgtatatcggcggcaatgtctgggaaggcgatcccgataaatgggacaagatgctgaatctcaacatcaacgccgccttccgttccgcccgcgccgtcatgcccgccatgatgaagcagaaaagcggcgatatcatcatgacaagctcgatcgcaggcatcgtcccgatcccggcggagccgatctacacggcttccaaacatgcggtgcaagccttcgctcacaccatacgccgccagttggccccgttcggcatccgcgtcggcgccatccagcccggcccggtcgtcacgcccttgctcaatgattgggaccccgagcgccttaaagccaatatcgaggctggcgccatgatgcagccttctgacgtcgccgaagccgtggttttcatgctgtcccgccgcaagggaacggtaatccgcgacttggttctgttaccccattctttcgacgtctaa

SEQ ID NO: 8 - protein, 242 aa, Kozakia baliensis
MTSPLQGKIAAITGGASGIGLECVRQIAASGATVYILDRDHQALDKAREELGERVHTIEVDLFRYETVDRAIETIVSEQGRIDILHVNAGAYIGGNVWEGDPDKWDKMLNLNINAAFRSARAVMPAMMKQKSGDIIMTSSIAGIVPIPAEPIYTASKHAVQAFAHTIRRQLAPFGIRVGAIQPGPVVTPLLNDWDPERLKANIEAGAMMQPSDVAEAVVFMLSRRKGTVIRDLVLLPHSFDV

SEQ ID NO: 9 - DNA, 729 bp, Providencia heimbachae
  source 1..729: mol_type = genomic DNA
atggctatatctctcgaaaacaacgtagctgcaattacaggtgccgcttcaggtatcggtctcgaatgtgcacgcacactgatcaaagcaggcgctaaagttgtcctcattgaccgagcagaagatagactaaatcaattggtcgcagaattaggtgaaaatgcaattccattagttatcgatttaatgaaaccagaacaagtcgatggcatgttagcgcgtattatcgaaaaggcaggcagattagatatctttcatgctaatgctggagcttacattggtgggcccgtagccgaaggcgatcccgatgtttgggataaagtcctaaatttaaatgttaatgccgcattccgctgtgttcgcgcagttctaccacactttatcgcacaaaagtcaggcgatattctattcaccagctctatcgctggtatggttcccgtaatttgggagcctatttacacggcatcaaaatttgcggttcaagcattcgttcattctactcgccgtcaggtttctgaacacggtgtccgtgttggtgctgtattacctggtcctgttgttactgcgttattagatgattggccaaaagaaaaacttgaagaagctttagctaacggtagtttaatgcaacccattgaagttgctgaggctgttctattcatgctgacgcgtccaagaaatattacaattcgcgatttagttattttacccaatagtgttgacctctaa

SEQ ID NO: 10 - protein, 242 aa, Providencia heimbachae
MAISLENNVAAITGAASGIGLECARTLIKAGAKVVLIDRAEDRLNQLVAELGENAIPLVIDLMKPEQVDGMLARIIEKAGRLDIFHANAGAYIGGPVAEGDPDVWDKVLNLNVNAAFRCVRAVLPHFIAQKSGDILFTSSIAGMVPVIWEPIYTASKFAVQAFVHSTRRQVSEHGVRVGAVLPGPVVTALLDDWPKEKLEEALANGSLMQPIEVAEAVLFMLTRPRNITIRDLVILPNSVDL

SEQ ID NO: 11 - DNA, 1206 bp, Starkeya novella
  source 1..1206: mol_type = genomic DNA
atggcgaaaatactttgcgttctctatgacgatccggtcgacggctacccgaagacctatgcgcgcgacgacctgccgaagatcgaccactatccgggcgggcagacgctgcccacgcccaaggcgatcgacttcacgccgggcgcgctgctcggctcggtctccggcgagctcggcctgcgcaaatacctggaagccaacggccataccttcgtcgtcacctccgataaggacggcccggattcggtgttcgagagggaactcgtcgacgccgacgtggtgatctcgcagcccttctggccggcctatctgacgcccgagcgcatcgccaaggcgaagaacctgaagctcgcgctcaccgccggcatcggctccgatcatgtcgatcttcagtcagctatcgaccgtggcatcactgtggccgaagtcacatattgcaactcgatcagcgtcgccgagcacgtggtgatgatgatcctcggcctggtacgaaactacattccctcgcatgactgggcgcgcaagggcggctggaacatagccgactgcgtagagcactcctacgacctcgagggcatgaccgtcggctcggtggccgccggccgcatcggcctcgccgtgctgcgccgcctcgcgccgttcgacgtgaagctgcactataccgaccgccaccgtctgccagaagcggtcgagaaggagctgggcctcgtctggcacgatacccgcgaggacatgtacccgcattgcgacgtggtcacgctcaacgtgccgctgcaccccgaaaccgagcacatgatcaatgacgagacgctgaagctgttcaagcgcggcgcctatatcgtcaacaccgcccgcggcaagctcgccgaccgcgacgccatcgtccgcgcgatcgagagcgggcagctcgcgggctatgccggcgacgtgtggttcccgcagccggctccgaaggaccacccctggcgcaccatgaagtgggaaggcatgacgccgcacatctccggcacctcgctctctgcccaggcgcgctacgcggcgggcacgcgcgagatcctcgaatgcttcttcgagggccggccgatccgcgacgagtacctgatcgtgcagggcggcgcgctcgccggcaccggcgcgcattcctactcgaagggcaatgcgaccggcggttcggaagaggccgcgaagttcaagaaggctggctga

SEQ ID NO: 12 - protein, 401 aa, Starkeya novella
MAKILCVLYDDPVDGYPKTYARDDLPKIDHYPGGQTLPTPKAIDFTPGALLGSVSGELGLRKYLEANGHTFVVTSDKDGPDSVFERELVDADVVISQPFWPAYLTPERIAKAKNLKLALTAGIGSDHVDLQSAIDRGITVAEVTYCNSISVAEHVVMMILGLVRNYIPSHDWARKGGWNIADCVEHSYDLEGMTVGSVAAGRIGLAVLRRLAPFDVKLHYTDRHRLPEAVEKELGLVWHDTREDMYPHCDVVTLNVPLHPETEHMINDETLKLFKRGAYIVNTARGKLADRDAIVRAIESGQLAGYAGDVWFPQPAPKDHPWRTMKWEGMTPHISGTSLSAQARYAAGTREILECFFEGRPIRDEYLIVQGGALAGTGAHSYSKGNATGGSEEAAKFKKAG